(19) United States
(12) Patent Application Publication
(10) Pub. No.: US 2022/0392059 A1
(43) Pub. Date: Dec. 8, 2022

(54) METHOD AND SYSTEM FOR REPRESENTATION LEARNING WITH SPARSE CONVOLUTION

(71) Applicant: SHENZHEN KEYA MEDICAL TECHNOLOGY CORPORATION, Shenzhen (CN)

(72) Inventors: Bin KONG, Charlotte, NC (US); Xin WANG, Seattle, WA (US); Youbing YIN, Kenmore, WA (US); Hao-Yu YANG, Seattle, WA (US); Yi LU, Seattle, WA (US); Xinyu GUO, Redmond, WA (US); Qi SONG, Seattle, WA (US)

(73) Assignee: SHENZHEN KEYA MEDICAL TECHNOLOGY CORPORATION, Shenzhen (CN)

(21) Appl. No.: 17/558,756

(22) Filed: Dec. 22, 2021

Related U.S. Application Data
(60) Provisional application No. 63/208,196, filed on Jun. 8, 2021.

(51) Int. Cl. G06T 7/00; G06V 10/40; G06V 10/764; G06V 10/82; G06V 10/77; G06T 7/73; G06N 3/08; G16H 30/40

(52) U.S. Cl. CPC G06T 7/0012; G06V 10/513; G06V 10/764; G06V 10/82; G06V 10/77; G06T 7/73; G06N 3/08; G16H 30/40; G06T 2207/20084; G06T 2207/30101; G06T 2207/30048; G06T 2207/20081

(57) ABSTRACT

Embodiments of the disclosure provide methods and systems for representation learning from a biomedical image with a sparse convolution. The exemplary system may include a communication interface configured to receive the biomedical image acquired by an image acquisition device. The system may further include at least one processor, configured to extract a structure of interest from the biomedical image. The at least one processor is also configured to generate sparse data representing the structure of interest and input features corresponding to the sparse data. The at least one processor is further configured to apply a sparse-convolution-based model to the biomedical image, the sparse data, and the input features to generate a biomedical processing result for the biomedical image. The sparse-convolution-based model performs one or more neural network operations including the sparse convolution on the sparse data and the input features.

### CROSS-REFERENCE TO RELATED APPLICATIONS

This application claims the benefit of priority to U.S. Provisional Application No. 63/208,196, filed on Jun. 8, 2021, the entire content of which is incorporated herein by reference.

### TECHNICAL FIELD

The present disclosure relates to methods and systems for processing biomedical images, and more particularly to efficient representation learning of biomedical images using sparse convolutions.

### BACKGROUND

Recent advances in the field of machine learning have made it possible to apply machine learning techniques to the analysis of biomedical images. For example, deep learning has been widely used as a representation learning method for extracting discriminative features from image data in an end-to-end manner. Convolution is the de-facto standard operation in many deep learning architectures such as Convolutional Neural Networks (CNNs) for extracting discriminative features from the image data. By virtue of local connectivity (each neuron is only connected to a small subset of neurons in the previous layer) and weight sharing (the same set of weights are used for extracting features for all spatial/temporal locations), traditional convolutions can be used to extract discriminative features from spatially/temporally dense data such as images and videos.

Some structures in biomedical image data can be more memory-efficiently represented in a sparse data format such as a point cloud, a graph, a list, etc. For instance, a coronary artery in a coronary computed tomography (CT) image can be more efficiently represented by a coronary artery tree. Despite of the recent advances in the machine learning field, this non-regular data (e.g., irregular sparse data) may present a unique challenge for learning representations with traditional convolutions.

One direction to address the challenge is to rasterize the sparse data to pixel/voxel grids so that the traditional convolutions can be applied. However, this method suffers from a computation burden caused by the problem of dimensionality, e.g., the computational cost grows exponentially with the dimensionality of the input data. For example, the computational cost with the traditional convolutions is generally orders of magnitude higher than that without the convolutions, as the convolution operation in each traditional convolution is conducted on each element of the input grid (e.g., the pixel/voxel grid). One way to alleviate this problem is to down-sample the input grid. Nevertheless, the down-sampling of the input grid may suffer from severe information loss, especially for small objects.

Another direction to leverage the sparsity of the non-regular data is to build convolutional layers such as Graph Convolutional Networks (GCNs) to directly learn representations from these sparsely represented data. These methods try to relax the definition of traditional convolutions to non-Euclidean spaces. However, these methods may waste a significant amount of time on structuring the irregular data. Additionally, the sparse data is generally associated with dense input image data in many applications (e.g., a coronary artery tree is associated with a coronary CT image), and a linking between the sparse data and the dense input image data is desirable.

Embodiments of the disclosure address the above problems by methods and systems for efficient representation learning of biomedical images with sparse convolutions.

### SUMMARY

Embodiments of methods and systems for processing biomedical images, and more particularly, for efficient representation learning of biomedical images with sparse convolutions, are disclosed.

In one aspect, embodiments of the disclosure provide a system for representation learning from a biomedical image with a sparse convolution. The exemplary system may include a communication interface configured to receive the biomedical image acquired by an image acquisition device. The system may further include at least one processor, configured to extract a structure of interest from the biomedical image. The at least one processor is also configured to generate sparse data representing the structure of interest and input features corresponding to the sparse data. The at least one processor is further configured to apply a sparse-convolution-based model to the biomedical image, the sparse data, and the input features to generate a biomedical processing result for the biomedical image. The sparse-convolution-based model performs one or more neural network operations including the sparse convolution on the sparse data and the input features.

In another aspect, embodiments of the disclosure also provide a method for representation learning from a biomedical image with a sparse convolution. The exemplary method may include receiving, at a communication interface, the biomedical image acquired by an image acquisition device. The method may also include extracting, by at least one processor, a structure of interest from the biomedical image. The method may further include generating, by the at least one processor, sparse data representing the structure of interest and input features corresponding to the sparse data. The method may additionally include applying, by the at least one processor, a sparse-convolution-based model to the biomedical image, the sparse data, and the input features to generate a biomedical processing result for the biomedical image. The sparse-convolution-based model performs one or more neural network operations including the sparse convolution on the sparse data and the input features.

In yet another aspect, embodiments of the disclosure further provide a non-transitory computer-readable medium having a computer program stored thereon. The computer program, when executed by at least one processor, performs a method for representation learning from a biomedical image with a sparse convolution. The exemplary method may include receiving the biomedical image acquired by an image acquisition device. The method may also include extracting a structure of interest from the biomedical image. The method may further include generating sparse data representing the structure of interest and input features corresponding to the sparse data. The method may additionally include applying a sparse-convolution-based model to the biomedical image, the sparse data, and the input features to generate a biomedical processing result for the biomedical image. The sparse-convolution-based model performs one or more neural network operations including the sparse convolution on the sparse data and the input features.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates a schematic diagram of an exemplary diagnostic image analysis system, according to certain embodiments of the disclosure.

FIG. 2A illustrates a schematic diagram of an exemplary framework overview of representation learning with at least a sparse convolution, according to certain embodiments of the disclosure.

FIG. 2B illustrates a schematic diagram of another exemplary framework overview of representation learning with at least a sparse convolution, according to certain embodiments of the disclosure.

FIG. 3 illustrates a schematic diagram of an exemplary image processing device, according to certain embodiments of the disclosure.

FIG. 4 is a graphical representation illustrating an exemplary convolutional operation n a sparse convolution, according to certain embodiments of the disclosure.

FIG. 5 is a graphical representation illustrating an exemplary application of a sparse-convolution-based model for a fractional flow reserve (FFR) prediction, according to certain embodiments of the disclosure.

FIG. 6 is a graphical representation illustrating another exemplary application of a sparse-convolution-based model for a branch label prediction, according to certain embodiments of the disclosure.

FIG. 7 is a flowchart of an exemplary method for training a sparse-convolution-based model, according to certain embodiments of the disclosure.

FIG. 8 is a flowchart of an exemplary method for representation learning with at least a sparse convolution, according to certain embodiments of the disclosure.

### DETAILED DESCRIPTION

Reference will now be made in detail to the exemplary embodiments, examples of which are illustrated in the accompanying drawings.

The disclosed methods and systems provide a sparse-convolution-based framework for efficient representation learning on biomedical images. The framework can determine sparse data from a biomedical image and generate a biomedical processing result by applying a sparse-convolution-based model to the biomedical image and the sparse data. A diagnostic output may be provided based on the biomedical processing result. The framework does not only leverage the sparsity of the sparse data effectively, but also aligns the sparse data with the associated biomedical image closely.

Consistent with the disclosure herein, the biomedical image can be a CT image, a Magnetic Resonance Imaging (MRI) image, an ultrasound image, or any other suitable biomedical image. Depending on the anatomical structure captured by the biomedical image, the sparse data can include a list of centerline points of a blood vessel (e.g., centerline points representing a coronary artery tree), a list of landmark points for a structure of interest, or the like. The biomedical processing result may include processing features for the biomedical image (e.g., processing features corresponding to the sparse data). Alternatively or additionally, the biomedical processing result may include a biomedical prediction result for the biomedical image. The biomedical prediction result can be a functional prediction result (e.g., FFR or instantaneous Wave-free Ratio (iFR) for a coronary artery in the form of:an FFR or iFR value at a centerline point, a relative FFR or iFR change at a centerline point compared to a neighboring point, etc.), a label prediction result (e.g., a label of a main branch in a blood vessel, a label of a side-branch in the blood vessel, etc. any other suitable biomedical prediction result (e.g., a disease type prediction result, a disease progression prediction result, a disease severity prediction result, a follow-up condition prediction result, etc.

In some embodiments, a diagnostic image analysis system may be configured to implement the sparse-convolution-based framework for efficient representation learning on biomedical images. For example, the diagnostic image analysis system may determine a structure of interest from a biomedical image and generate sparse data representing the structure of interest. The diagnostic image analysis system may also generate input features corresponding to the sparse data. The sparse data and the input features may form an input sparse tensor. The diagnostic image analysis system may apply a sparse-convolution-based model to the biomedical image and the input sparse tensor to generate a biomedical processing result for the biomedical image. In some embodiments, the sparse-convolution-based model may perform one or more sparse convolutions and/or one or more neural network operations on the input sparse tensor and the biomedical image to generate the biomedical processing result. The diagnostic image analysis system may further provide a diagnostic output based on the biomedical processing result. The representations (e.g., the processing features) extracted by the sparse-convolution-based model may also be used by other down-streaming devices and systems such as follow-up condition prediction.

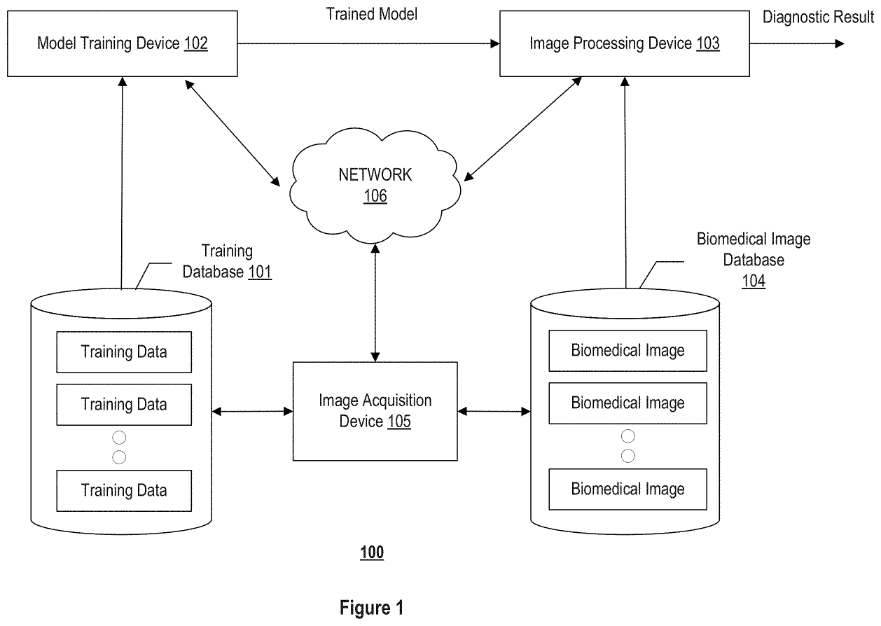

FIG. 1 illustrates an exemplary diagnostic image analysis system 100, according to some embodiments of the present disclosure. Consistent with the present disclosure, diagnostic image analysis system 100 may be configured to analyze a biomedical image acquired by an image acquisition device 105 and perform a diagnostic prediction based on the image analysis. In some embodiments, image acquisition device 105 may be a CT scanner that acquires 2D or 3D CT images. For example, image acquisition device 105 may be a 3D multi-detector row CT scanner for volumetric CT scans. In some embodiments, image acquisition device 105 may use one or more other imaging modalities, including, e.g., Magnetic Resonance Imaging (MRI), functional MRI (e.g., fMRI, DCE-MRI and diffusion MRI), Positron Emission Tomography (PET), Single-Photon Emission Computed Tomography (SPECT), X-ray, Optical Coherence Tomography (OCT), fluorescence imaging, ultrasound imaging, radiotherapy portal imaging, or the like.

In some embodiments, image acquisition device 105 may capture images including at least one anatomical structure or organ, such as a heart, a liver, a lung, or a thorax. In some embodiments, each volumetric CT exam may include 20-1094 CT slices with a varying slice-thickness from 0.25 mm to 5 mm. The reconstruction matrix may have 512×512 pixels with in-plane pixel spatial resolution from 0.29×0.29 mm² to 0.98×0.98 mm².

As shown in FIG. 1, diagnostic image analysis system 100 may include components for performing two phases, a training phase and a prediction phase. The prediction phase may also be referred to as an inference phase. To perform the training phase, diagnostic image analysis system 100 may include a training database 101 and a model training device 102. To Perform the prediction phase, diagnostic image analysis system 100 may include an image processing device 103 and a biomedical image database 104. In some embodiments, diagnostic image analysis system 100 may include more or less of the components shown in FIG. 1. For example, when a diagnosis model (e.g., a sparse-convolution-based model) for providing the diagnostic prediction based on the biomedical images is pre-trained and provided, diagnostic image analysis system 100 may include only image processing device 103 and biomedical image database 104.

Diagnostic image analysis system 100 may optionally include a network 106 to facilitate the communication among the various components of diagnostic image analysis system 100, such as databases 101 and 104, devices 102, 103, and 105. For example, network 106 may be a local area network (LAN), a wireless network, a cloud computing environment (e.g., software as a service, platform as a service, infrastructure as a service), a client-server, a wide area network (WAN), etc. In some embodiments, network 106 may be replaced by wired data communication systems or devices.

In some embodiments, the various components of diagnostic image analysis system 100 may be remote from each other or in different locations, and be connected through network 106 as shown in FIG. 1. In some alternative embodiments, certain components of diagnostic image analysis system 100 may be located on the same site or inside one device. For example, training database 101 may be located on-site with or be part of model training device 102. As another example, model training device 102 and image processing device 103 may be inside the same computer or processing device.

Model training device 102 may use the training data received from training database 101 to train a diagnosis model (e.g., a sparse-convolution-based model) for analyzing a biomedical image received from, e.g., biomedical image database 104, in order to provide a diagnostic prediction. As shown in FIG. 1, model training device 102 may communicate with training database 101 to receive one or more sets of training data (e.g., one or more training datasets). In certain embodiments, each training dataset may include ground truth of a biomedical result which may include at least one of an FFR or iFR result of a blood vessel, branch labels of a blood vessel, patient information, testing results, ongoing treatment information, or the like. The FFR or iFR result of a blood vessel may include, for example, an FFR or iFR value at each centerline point of the blood vessel, a relative FFR or iFR change at a centerline point compared with a neighboring point, etc. The branch labels of a blood vessel may include, for example, a label of a main branch in the blood vessel (e.g., a right coronary artery, a left anterior descending, a left circumflex, etc.) or a label of a side-branch in the blood vessel (e.g., a right posterior lateral branch, a right posterior descending artery, diagonal arteries, obtuse marginal arteries, a left posterior lateral branch, etc.).

Training images stored in training database 101 may be obtained from a biomedical image database containing previously acquired images of anatomical structures. In some embodiments, the biomedical image may be processed by model training device 102 to identify specific diseases, anatomical structures, support structures, and other items or biomedical results. The biomedical processing results (e.g., biomedical prediction results) outputted from the model are compared with an initial diseases/finding probability analysis, and based on the difference, the model parameters are improved/optimized by model training device 102. For example, an initial diseases/findings probability analysis may be performed or verified by experts.

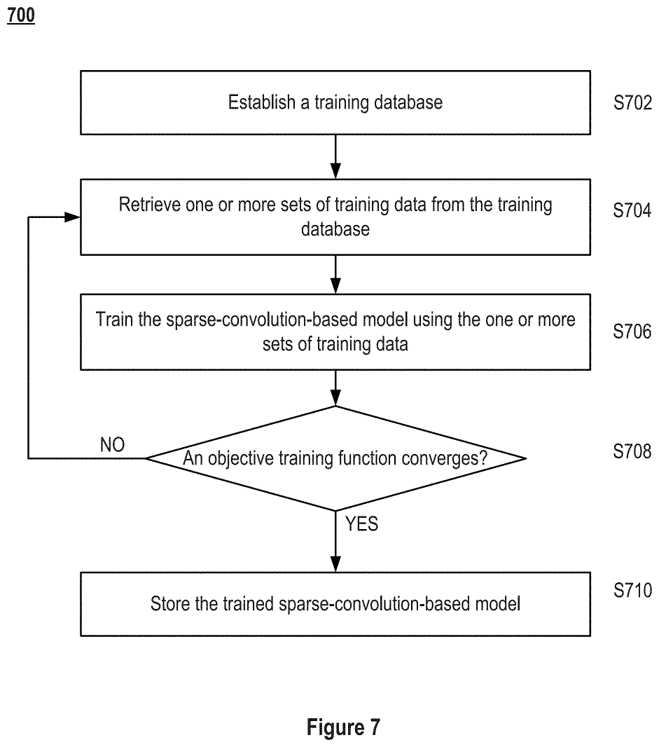

In some embodiments, model training device 102 may be configured to train a diagnosis model (e.g., a sparse-convolution-based model) in an end-to-end manner using optimization methods such as stochastic gradient descent (SGD), root mean square prop (RMSProp), adaptive moment estimation (Adam), or the like. During the training phase, annotated training datasets with the ground truth values (or ground truth annotations) can be retrieved from training database 101 to train the diagnosis model. As a result, a mapping between the inputs and the ground truth values (or ground truth annotations) is learned by finding the best fit between the biomedical processing results (e.g., processing features and/or biomedical prediction results) and the ground truth values (or ground truth annotations) over the training datasets using the diagnosis model. Model training device 102 is further described below in more detail with reference to FIG. 7.

In some embodiments, the training phase may be performed “online” or “offline,” An “online” training refers to performing the training phase contemporarily with the prediction phase, e.g., learning the model in real-time just prior to analyzing a biomedical image. An “line” training may have the benefit to obtain a most updated learning model based on the training data that is then available. However, an “online” training may be computational costive to perform and may not always be possible if the training data is large and/or the model is complicate. Consistent with the present disclosure, an “offline” training is used where the training phase is performed separately from the prediction phase. The learned model trained offline is saved and reused for analyzing biomedical images.

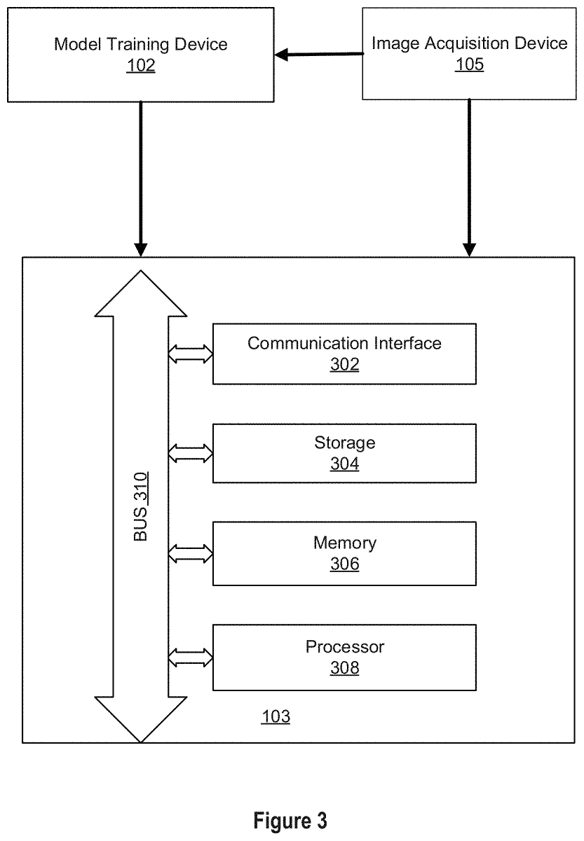

Model training device 102 may be implemented with hardware specially programmed by software that performs the training process. For example, model training device 102 may include a processor and a non-transitory computer-readable medium (discussed in detail in connection with FIG. 3) The processor may conduct the training by performing instructions of a training process stored in the computer-readable medium. Model training device 102 may additionally include input and output interfaces to communicate with training database 101, network 106, and/or a user interface (not shown). The user interface may be used for selecting sets of training data, adjusting one or more parameters of the training process, selecting or modifying a framework of the learning model, and/or manually or semi-automatically providing biomedical processing results associated with an image for raining.

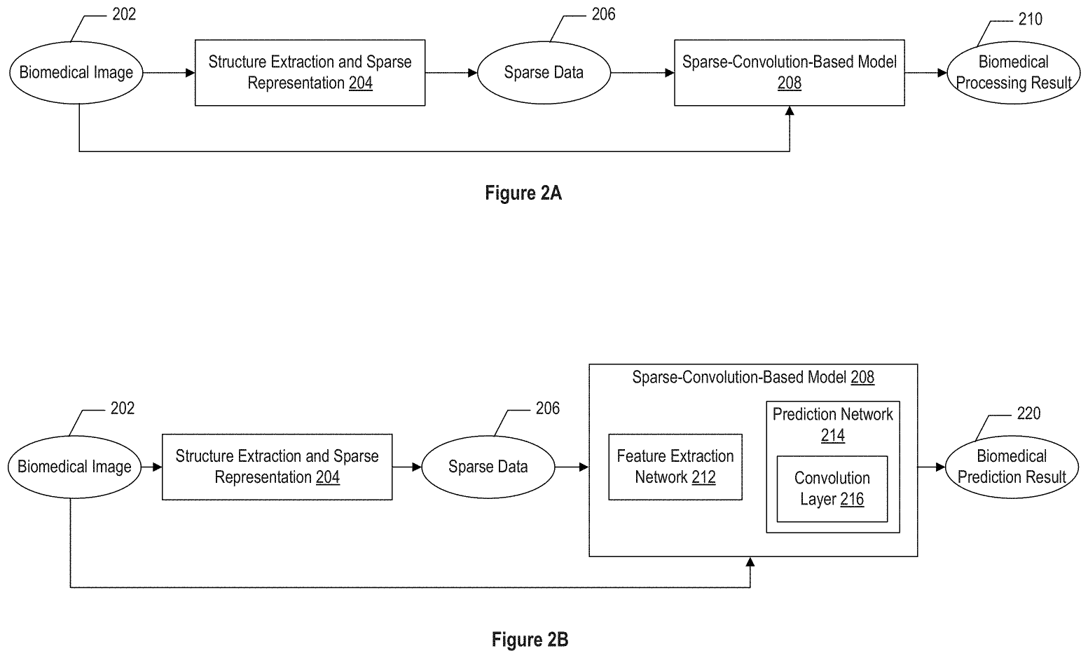

Consistent with some embodiments, the trained diagnosis model (e.g., the trained sparse-convolution-based model) may be used by image processing device 103 to analyze new biomedical images for diagnosis purpose. Image processing device 103 may receive the diagnosis model, e.g., a sparse-convolution-based model 208 shown in FIGS. 2A-2B that will be described in detail later, from model training device 102. Image processing device 103 may include a processor and a non-transitory computer-readable medium (discussed in detail in connection with FIG. 3). The processor may perform instructions of a medical diagnostic image analysis program stored in the medium. Image processing device 103 may additionally include input and output interfaces (discussed in detail in connection with FIG. 3) to communicate with biomedical image database 104, network 106, and/or a user interface (not shown). The user interface may be used for selecting biomedical images for analysis, initiating the analysis process, displaying the diagnostic results, or the like.

Image processing device 103 may communicate with biomedical image database 104 to receive biomedical images. In some embodiments, the biomedical images stored in biomedical image database 104 may include 2D or 3D (or even higher dimensional) images (e.g., 2D or 3D cardiac CT images) from one or more underlying subjects (e.g., patients susceptible to heart diseases). The biomedical images may be acquired by image acquisition devices 105. Image processing device 103 may extract a structure of interest from a biomedical image and generate sparse data representing the structure of interest. Image processing device 103 may also generate input features corresponding to the sparse data. The sparse data and the input features corresponding to the sparse data may form an input sparse tensor.

For example, image processing device 103 may extract a centerline of a coronary artery in a cardiac image, and generate sparse data representing centerline points of the coronary artery. The centerline points of the coronary artery may be generated by discretizing the centerline of the coronary artery, and may form a coronary artery tree. Image processing device 103 may also generate an input feature for each centerline point of the coronary artery. Image processing device 103 may form an input sparse tensor including (1) the sparse data representing the centerline points of the coronary artery and (2) the input features corresponding the centerline points of the coronary artery.

Image processing device 103 may apply a sparse-convolution-based model to the biomedical image and the input sparse tensor to generate a biomedical processing result for the biomedical image, Image processing device 103 may provide a diagnostic output based on at least one of the biomedical processing result, patient information, testing results, ongoing treatment information, etc. Image processing device 103 is further described below in more detail with reference to FIGS. 2A-2B, 3-6, and 8.

FIG. 2A illustrates a schematic diagram of an exemplary framework overview of representation learning with at least a sparse convolution, according to certain embodiments of the disclosure. Initially, image processing device 103 may obtain a biomedical image 202 to be processed from biomedical image database 104. Image processing device 103 may perform a structure extraction and sparse representation operation 204 on biomedical image 202 to generate sparse data 206. In some embodiments, sparse data 206 may include a set of elements sparsely representing the structure of interest. Sparse data 206 may also indicate a set of coordinates for the set of elements, respectively. The set of coordinates may be used to identify a set of locations for the set of elements within biomedical image 202, respectively.

In some embodiments, sparse data 206 may be expressed in any other suitable sparse format including, e.g., masks, graphs, trees, etc., that can be used to sparsely represent the structure of interest. For example, a Graph Convolutional Neural Network (GCN) can be an exemplary way to structure biomedical image 202 to generate sparse data 206, and can effectively utilize the sparsity of sparse data 206. In some embodiments, the sparse convolution can be a convolution conducted in a specified subset of elements of biomedical image 202, instead of all the elements of biomedical image 202, Sparsity data 206 can be structured in different ways such as masks, trees, graphs, a list (or a set) of coordinates, etc.

Specifically, image processing device 103 may extract a structure of interest from biomedical image 202. Image processing device 103 may generate sparse data 206 representing the structure of interest by: determining a set of elements that can sparsely represent the structure of interest from biomedical image 202; and determining a set of coordinates for the set of elements from biomedical image 202, respectively. Each element of sparse data 206 may be associated with one or more coordinates (e.g., x and y coordinate values) that may be used to identify a location of the element within biomedical image 202. For example, image processing device 103 may discretize the extracted structure of interest into a set of key points. Each element of sparse data 206 may be used to represent a corresponding key point in the structure of interest, and may have the same coordinates as the corresponding key point within biomedical image 202. In this case, the structure of interest can be efficiently represented by sparse data 206 (e.g., the structure of interest is converted to a sparse format represented by sparse data 206).

For example, image processing device 103 may segment biomedical image 202 to obtain a tree structure of a blood vessel. Image processing device 103 may identify a centerline of the tree structure a centerline of a coronary artery). Then, image processing device 103 may discretize the centerline of the tree structure into a set of centerline points, and may generate sparse data 206 that sparsely represents the centerline of the tree structure. Sparse data 206 may include a set of elements corresponding to the set of centerline points, with each element representing a corresponding centerline point in the tree structure and having the same coordinates as the corresponding centerline point within biomedical image 202. Consistent with the present disclosure, a “centerline” may be a skeleton line of the structure of interest, and may generally track the structure of interest, including one or more “trunks” and/or one or more “branches” of the structure of interest.

In another example, image processing device 103 may segment biomedical image 202 to obtain an object of interest (e.g., a lung, a liver, etc.). Image processing device 103 may identify a set of landmarks from the object of interest, and may generate sparse data 206 that represents the set of landmarks associated with the object of interest. Sparse data 206 may include a set of elements corresponding to the set of landmarks, with each element representing a corresponding landmark in the object of interest and having the same coordinates as the corresponding landmark within biomedical image 202.

Next, image processing device 103 may feed sparse data 206 and biomedical image 202 into a sparse-convolution-based model 208 to generate a biomedical processing result 210, as described below in more detail. In some embodiments, sparse-convolution-based model 208 may be trained by model training device 102 online or offline, and may be used to generate the biomedical processing result after being trained.

In some embodiments, image processing device 103 may receive or automatically generate a set of input features corresponding to the set of elements in sparse data 206, with each input feature corresponding to an element of sparse data 206. In some examples, biomedical image 202 may be a raw biomedical image, and the set of input features may be directly from or derived from the raw biomedical image. In some alternative examples, biomedical image 202 may be a processed biomedical image that is obtained by performing one or more pre-processing operations on the raw biomedical image. For example, image processing device 103 may perform a masking operation, an operation with different mage intensity transformation, or any other suitable pre-processing operation on the raw biomedical image to obtain the processed biomedical image. Image processing device 103 may then generate the set of input features from the processed biomedical image.

In some embodiments, the set of input features can be hand-crafted (i.e., designed by users). Alternatively, the set of input features can be automatically learned by a feature learning model (e.g., a neural network such as a feature extraction network 212 shown in 2B). Feature extraction network 212 is described below in more detail with reference to FIG. 2B.

In some embodiments, biomedical processing result 210 may include processing features corresponding to sparse data 206. Image processing device 103 may apply sparse-convolution-based model 208 to extract the processing features from biomedical image 202 based on sparse data 206 and the input features corresponding to the sparse data. The processing features can be features outputted by sparse-convolution-based model 208. For example, sparse-convolution-based model 208 may include a sparse-convolution-based feature extraction network that extracts the processing features from biomedical image 202 based on sparse data 206 and the input features. The sparse-convolution-based feature extraction network can be any suitable feature-extraction network, where one or more sparse convolutions are applied in the network to replace one or more traditional convolutions in the network.

Image processing device 103 may further apply sparse-convolution-based model 208 to generate a biomedical prediction result from biomedical image 202 based on sparse data 206 and the processing features. For example, sparse-convolution-based model 208 may further include a sparse-convolution-based prediction network (e.g., a RNN network) that generates the biomedical prediction result from biomedical image 202 based on sparse data 206 and the processing features, where one or more sparse convolutions are applied in the network to replace one or more traditional convolutions in the network.

Alternatively, image processing device 103 may further apply another suitable model (e.g., a prediction model such as a RNN model) to generate a biomedical prediction result from biomedical image 202 based on sparse data 206 and the processing features. The prediction model can be a sparse-convolution-based prediction model (e.g., a sparse-convolution-based prediction network). Depending on the actual application needs, the prediction model can be separate from or incorporated into sparse-convolution-based model 208.

In some embodiments, biomedical processing result 210 may include a biomedical prediction result. Image processing device 103 may apply sparse-convolution-based model 208 to generate the biomedical prediction result from biomedical image 202 based on sparse data 206 and the input features corresponding to sparse data 206.

In some embodiments, image processing device 103 may generate an input sparse tensor including sparse data 206 representing the structure of interest and the set of input features corresponding to sparse data 206. Image processing device 103 may apply sparse-convolution-based model 208 to biomedical image 202 and the input sparse tensor to generate biomedical processing result 210 for biomedical image 202. Sparse-convolution-based model 208 may perform one or more sparse convolutions and/or any other suitable neural network operations (e.g., activation functions, pooling operations, etc.) on the input sparse tensor and biomedical image 202. In this case, image processing device 103 may use sparse-convolution-based model 208 to maximally leverage the sparsity of sparse data 206.

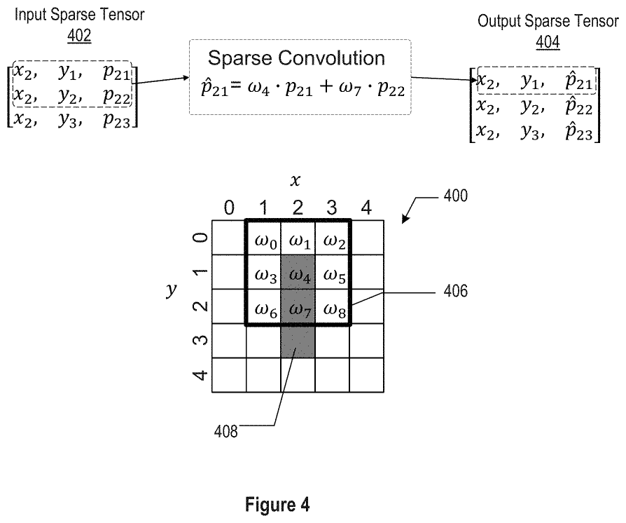

Sparse-convolution-based model 208 may be a deep learning model that utilizes sparse convolutions to replace traditional convolutions in the model. For example, sparse-convolution-based model 208 may include a Convolutional Neural Network (CNN), a Multilayer Perceptron (MLP), a Fully Convolutional Network (FCN), a tree structured recurrent neural network (RNN), a graph network, or any other suitable deep learning network, where one or more sparse convolutions are applied in the network to replace one or more traditional convolutions in the network. The sparse convolution as well as a comparison between the sparse convolution and the traditional convolution is described below in more detail with reference to FIG. 4.

For example, the structure of interest extracted from biomedical image 202 may be a centerline of a blood vessel, and sparse data 206 may include centerline points of the blood vessel. For example, biomedical image 202 can be a cardiac image, and the blood vessel can be a coronary artery, and accordingly, the centerline points may form a coronary artery tree of the blood vessel. Image processing device 103 may generate an input sparse tensor including (1) a set of elements corresponding to the centerline points of the blood vessel and (2) a set of input features corresponding to the centerline points of the blood vessel.

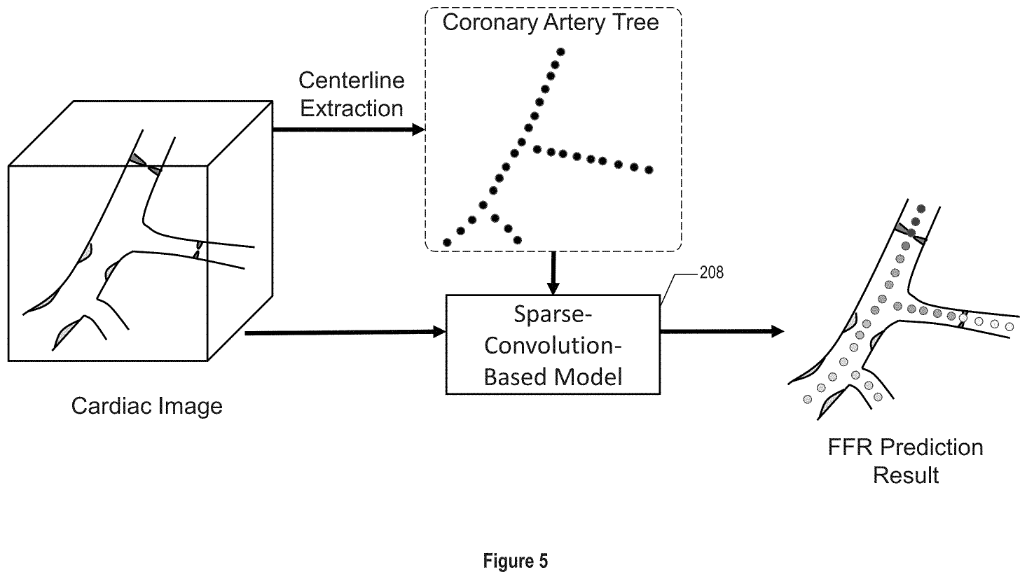

Image processing device 103 may apply sparse-convolution-based model 208 to biomedical image 202 and the input sparse tensor to generate an FFR prediction result for the blood vessel, as described below in more detail with reference to FIG. 5. The FFR prediction result may include an FFR value at a centerline point, or a relative FFR change at a centerline point compared to a neighboring point of the blood vessel.

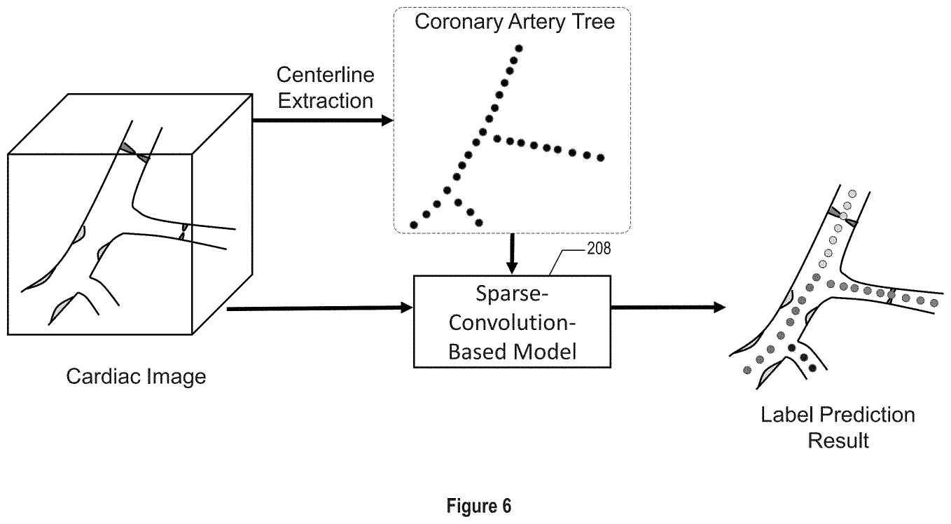

Alternatively or additionally, image processing device 103 may apply sparse-convolution-based model 208 to biomedical image 202 and the input sparse tensor to generate a label prediction result for the blood vessel, as described below in more detail with reference to FIG. 6. The label prediction result may include at least one of a label of a main branch or a label of a side-branch in the blood vessel. Exemplary labeling of a main branch in the blood vessel may include, but is not limited to, at least one of the following: a label of a right coronary artery, a label of a left anterior descending, or a label of a left circumflex for the blood vessel. Exemplary labeling of a side-branch in the blood vessel may include, but is not limited to, at least one of the following: a label of a right posterior lateral branch, a label of a right posterior descending artery, a label of two diagonal arteries, a label of two obtuse marginal arteries, or a label of a left posterior lateral branch.

FIG. 2B illustrates a schematic diagram of another exemplary framework overview of representation learning with at least a sparse convolution, according to certain embodiments of the disclosure. The exemplary framework in FIG. 2B may have elements like those of the exemplary framework in FIG. 2A, and the similar description will not be repeated here. Referring to FIG. 2B, sparse-convolution-based model 208 may include feature extraction network 212 and a prediction network 214. In some embodiments, feature extraction network 212 and prediction network 214 can be trained in the training phase of sparse-convolution-based model 208. For example, model training device 102 may train feature extraction network 212 and prediction network 214 together or separately using training data obtained from training database 101.

Feature extraction network 212 can be any neural network for extracting the set of input features for the set of elements in sparse data 206. For example, feature extraction network 212 may include pairs of convolutional layers and pooling layers to extract the set of input features for the set of elements in sparse data 206 from biomedical image 202. In some embodiments, feature extraction network 212 can be a sparse-convolution-based feature extraction network such that sparse convolutions (rather than traditional convolutions) are applied in its convolution layers. For example, feature extraction network 212 may include a CNN, an FCN, or any other suitable neural network, where one or more sparse convolutions are applied in the network to replace one or more traditional convolutions in the network.

In some embodiments, image processing device 103 may apply feature extraction network 212 to extract the set of input features from biomedical image 202 based on a set of coordinates corresponding to the set of elements in sparse data 206. For example, for each element in sparse data 206, image processing device 103 may determine one or more coordinates associated with the element to identify a location of the element of sparse data 206 in biomedical image 202, Image processing device 103 may apply feature extraction network 212 to biomedical image 202 to obtain a feature of an image element (e.g., a pixel element) at the identified location of biomedical image 202. Image processing device 103 may use the feature of the image element at the identified location of biomedical image 202 as an input feature for the element of sparse data 206.

Prediction network 214 can be any neural network for generating a biomedical prediction result 220. For example, prediction network 214 may include one or more convolutional layers 216 and any other suitable neural network layers (e.g., activation functions, pooling layers, etc.) to generate biomedical prediction result 220 from biomedical image 202 and the input sparse tensor. In some embodiments, prediction network 214 can be a sparse-convolution-based prediction network such that sparse convolutions (rather than traditional convolutions) are applied in its convolution layers 216. For example, prediction network 214 may include a CNN, a MLP, or an FCN, where one or more sparse convolutions are applied in the network to replace one or more traditional convolutions in the network. In another example, prediction network 214 can include a tree structured RNN, a graph network, or any other suitable deep learning network to generate a functional prediction result (e.g., an FFR prediction result, an iFR prediction result, etc.).

In some embodiments, image processing device 103 may apply prediction network 214 to biomedical image 202 and the input sparse tensor to generate an FFR prediction result for a blood vessel in biomedical image 202. In some embodiments, image processing device 103 may apply prediction network 214 to biomedical image 202 and the input sparse tensor to generate a label prediction result for the blood vessel.

Systems and methods of the present disclosure may be implemented using a computer system, such as one shown in FIG. 3. In some embodiments, image processing device 103 may be a dedicated device or a general-purpose device. For example, image processing device 103 may be a computer customized for a hospital for processing image data acquisition and image data processing tasks, or a server cloud environment. Image processing device 103 may include one or more processor(s) 308, one or more storage device(s) 304, and one or more memory device(s) 306. Processor(s) 308, storage device(s) 304, and memory device(s) 306 may be configured in a centralized or a distributed manner. Image processing device 103 may also include a biomedical image database (optionally stored in storage device 304 or in a remote storage), an input/output device (not shown, but which may include a touch screen, keyboard, mouse, speakers/microphone, or the like), a network interface such as communication interface 302, a display (not shown, but which may be a cathode ray tube (CRT) or liquid crystal display (LCD) or the like), and other accessories or peripheral devices. The various elements of image processing device 103 may be connected by a bus 310, which may be a physical and/or logical bus in a computing device or among computing devices.

Processor 308 may be a processing device that includes one or more general processing devices, such as a microprocessor, a central processing unit (CPU), a graphics processing unit (GPU), and the like. More specifically, processor 308 may be a complex instruction set computing (CISC) microprocessor, a reduced instruction set computing (RISC) microprocessor, a very long instruction word (VLIW) microprocessor, a processor running other instruction sets, or a processor that runs a combination of instruction sets. Processor 308 may also be one or more dedicated processing devices such as application specific integrated circuits (ASICs), field programmable gate arrays (FPGAs), digital signal processors (DSPs), system-on-chip (SoCs), and the like.

Processor 308 may be communicatively coupled to storage device 304/memory device 306 and configured to execute computer-executable instructions stored therein. For example, as illustrated in FIG. 3, bus 310 may be used, although a logical or physical star or ring topology would be examples of other acceptable communication topologies. Storage device 304/memory device 306 may include a read only memory (ROM), a flash memory, random access memory (RAM), a static memory, a volatile or non-volatile, magnetic, semiconductor, tape, optical, removable, nonremovable, or other type of storage device or tangible (e.g., non-transitory) computer readable medium. In some embodiments, storage device 304 may store computer-executable instructions of one or more processing programs, learning models/networks used for the processing (e.g., model 208, network 212, network 214), and data (e.g., sparse data, an input sparse tensor, a biomedical processing result) generated when a computer program is executed. The data may be read from storage device 304 one by one or simultaneously and stored in memory device 306. Processor 308 may execute the processing program to implement each step of the methods described below, Processor 308 may also send/receive medical data to/from storage device 304/memory device 306 via bus 310.

Image processing device 103 may also include one or more digital and/or analog communication (input/output) devices, not illustrated in FIG. 3. For example, the input/output device may include a keyboard and a mouse or trackball that allow a user to provide input. Image processing device 103 may further include a network interface, illustrated as communication interface 302, such as a network adapter, a cable connector, a serial connector, a USB connector, a parallel connector, a high-speed data transmission adapter such as optical fiber, USB 3.0, lightning, a wireless network adapter such as a WiFi adapter, or a telecommunication (3G, 4G/LTE, etc.) adapter and the like. Image processing device 103 may be connected to a network through the network interface. Image processing device 103 may further include a display, as mentioned above. In some embodiments, the display may be any display device suitable for displaying a medical image and its diagnostic results. For example, the image display may be an LCD, a CRT, or an LED display.

Image processing device 103 may be connected, wired or wirelessly, to model training device 102 and image acquisition device 105 as discussed above with reference to FIG. 1, Other implementations are also possible,

FIG. 4 is a graphical representation illustrating an exemplary convolutional operation in a sparse convolution, according to certain embodiments of the disclosure. The sparse convolution aims to leverage the sparsity of the sparse data in the maximum extent. More specifically, given an input sparse tensor S={(cᵢ, pᵢ)}_{i×0}^{N-1}, a convolutional operation in the sparse convolution can be defined as follows:

\hat{p}ᵢ= [symbol] ωⱼ·pⱼ. (1)

Herein, cᵢ denotes coordinates of an i^{th} element of the sparse data (or equivalently, an i^{th} element of the input sparse tensor), and may include an x coordinate value and ay coordinate value of the i^{th} element. pᵢ denotes an input feature of the i^{th} element. N denotes a total number of elements in the structure of interest (or equivalently, a total number of elements in the input sparse tensor). ωⱼ denotes a j^{th} element (or a j^{th} weight) of a convolutional kernel. \hat{p}ᵢ denotes an output of the convolutional operation (e.g., an updated feature) for the i^{th} element [symbol] ᵢ denotes a collection of indices that are in the vicinity of the i^{th} element [symbol] ᵢ can be used to identify a collection of vicinity elements for the i^{th} element, which includes the i^{th} element itself and a group of neighboring elements adjacent to the element from the input sparse tensor S.

By way of examples, FIG. 4 illustrates an exemplary biomedical map 400 having elements (xₗ, yₖ) arranged in an array, with 0≤l≤4 and 0≤k≤4. Each element (xₗ, yₖ) is associated with an input feature pₗₖ. Biomedical map 400 can be a biomedical image or a feature map obtained during the processing of the biomedical image by sparse-convolution-based model 208.

When a traditional convolution is performed on biomedical map 400, an updated feature \hat{p}ₗₖ for the element (xₗ, yₖ) can be generated by iterating all the input features surrounding the element (xₗ, yₖ). For example, for a 3×3 convolutional kernel 406, an updated feature \hat{p}₂₁ for the element (x₂, y₁p can be generated by iterating the input features of all the elements within convolutional kernel 406, where the element (x₂, y₁) is centered in convolutional kernel 406. That is, the updated feature \hat{p}₂₁ can be calculated as:

\hat{p}₂₁=ω₀·p₁₀+ω₁·p₂₀+ω₂·p₃₀+ω₃·p₁₁+ω₄·p₂₁+ω₅·p₃₁+ω₆·p₁₂+ω₇·p₂₂+ω₈·p₂₃. (2)

It can be seen from the above expression (2) that in the traditional convolution, an updated feature for an element is calculated by iterating through all elements surrounding the element in the rasterized biomedical map. That is; the updated feature for the element is calculated by iterating through all elements covered by the convolutional kernel (e.g., the 3×3 kernel 406 shown in FIG. 4).

On the other hand, a sparse convolution can be performed on biomedical map 400 based on an input sparse tensor 402. Input sparse tensor 402 only includes three elements with coordinates (x₂, y₁), (x₂, y₂); and (x₂, y₃) to represent an exemplary structure of interest; as shown in a shaded area 408 in FIG. 4. Input sparse tensor 402 also includes three input features p₂₁, p₂₂, and p₂₃ for the three elements, respectively. An output sparse tensor 404 may be generated by the sparse convolution according to the above expression (1). Output sparse tensor 404 includes three updated features \hat{p}₂₁, \hat{P}₂₂, and \hat{p}₂₃ for the three elements (x₂, y₁); (x₂, y₂), and (x₂, y₃) respectively.

Specifically, with respect to each element in input sparse tensor 402, a collection of vicinity elements can be determined for the element from input sparse tensor 402. The collection of vicinity elements may include the element itself and a group of neighboring elements adjacent to the element from input sparse tensor 402. A collection of input features associated with the collection of vicinity elements can also be deter mined from input sparse tensor 402. An updated feature of the element can be determined based on the collection of input features associated with the collection of vicinity elements. For example; the updated feature of the element can be equal to a weighted sum of the collection of input features associated with the collection of vicinity elements.

For example, with respect to the element with the coordinates (x₂, y₁) as shown in shaded area 408, only the element with coordinates (x₂, y₂) from input sparse tensor 402 is adjacent to the element with the coordinates (x₂, y₁), whereas the element with coordinates (x₂, y₃) from input sparse tensor 402 is not adjacent to the element with the coordinates (x₂, y₁). Thus, a collection of vicinity elements for the element h the coordinates (x₂, y₁) can be determined to include the element itself and the element with coordinates (x₂, y₂). The input features for the collection of vicinity elements include p₂₁ and p₂₂. Then, the updated feature \hat{P}₁₂ for the element with the coordinates (x₂, y₁p can be calculated according to the above expression (1) as follows:

\hat{P}₁₂=ω₄·p₂₁+ω₇·p₂₂. (3)

Expression (3) shows that in the sparse convolution, an updated feature for an element is calculated by only iterating through a collection of vicinity elements in the input sparse tensor S. This is different from the traditional convolution, which generates the updated feature for the element by iterating through all elements covered by the convolutional kernel as described above. The number of elements included in the collection of vicinity elements is much less than the total number of elements covered by the convolutional kernel (e.g., 50%, 60%, or 70% less). Additionally, the updated features are calculated for every coordinate (i.e., (x₀, y₀), (x₀, y₁), . . . , (x₄, y₄), a total number of 25 elements) of the input age in the traditional convolution, whereas the sparse convolution only updates the features at the specified coordinates (i.e., (x₁, y₂), (x₂, y₂), and (x₃, y₂) in this example). As a result, a computation of the sparse convolution is orders of magnitude more efficient than that of the traditional convolution.

Comparing the sparse convolution with the traditional convolution, the traditional convolution is equivalent to the sparse convolution if the following condition is satisfied: for each element that is covered by the convolution kernel of the traditional convolution but not included in the input sparse tensor of the sparse convolution, a corresponding element (or weight) of the convolutional kernel is zero. For example, with respect to the above expression (2) for the traditional convolution, if ωⱼ=0 for j=0, 1, 2, 5, 6, and 8 (which correspond to the elements covered by convolutional kernel 406 but not included in input sparse tensor 402), the above expression (2) becomes the same as the above expression (3) for the sparse convolution.

Consistent with certain embodiments of the present disclosure, sparse-convolution-based model 208 may include a series of sparse convolutions. If there is another sparse convolution following the sparse convolution shown in FIG. 4, output sparse tensor 404 can be treated as an input sparse tensor for the next sparse convolution. Additionally or alternatively, other neural network operations (e.g., a pooling operation or any other suitable operation) can be performed on output sparse tensor 404. Input sparse tensor 402 can be an input sparse tensor derived directly from an original biomedical image. Alternatively, input sparse tensor 402 can be an output sparse tensor obtained from a previous sparse convolution preceding the sparse convolution shown in FIG. 4.

FIG. 5 is a graphical representation illustrating an exemplary application of sparse-convolution-based model 208 for an FFR prediction, according to certain embodiments of the disclosure. In this example, a biomedical image can be a cardiac image, and a structure of interest extracted from the cardiac image may be a centerline of a blood vessel such as a centerline of a coronary artery. Sparse data of the cardiac image may include centerline points of the coronary artery. The centerline points may form a coronary artery tree, Sparse-convolution-based model 208 can be applied to the cardiac image and the sparse data to generate an FFR prediction result.

For example, image processing device 103 may generate an input sparse tensor including the sparse data (e.g., a set of elements corresponding to the centerline points of the coronary artery) and a set of input features corresponding to the centerline points of the coronary artery. As only features of the centerline points are needed, the sparse convolution can also be used to speed up the feature extraction from the cardiac image in order to obtain the input features. In some embodiments, the input features may be directly derived from a raw cardiac image or a processed cardiac image. The processed cardiac image can be obtained by performing a mask operation, a pre-processing operation with different image intensity transformations, or any other suitable pre-processing operation on the raw cardiac image. The input features can be hand-crafted (i.e., designed by humans) or automatically learned by models (e.g., feature extraction network 212) from the cardiac image. The input sparse tensor can be formed to include the sparse data and the input features corresponding to the sparse data.

Subsequently, sparse-convolution-based model 208 (e.g., prediction network 214 of model 208) may be applied to the cardiac image and the input sparse tensor to generate the FFR prediction result. The FFR prediction result may include an FFR value at each centerline point, a relative FFR change of each centerline point compared with its neighboring point, etc. Prediction network 214 may be a CNN, an MIT, an FCN, a tree structured RNN, a graph network, etc. As the sparse convolutions in model 208 are only conducted on the coronary artery region of the cardiac image, the computation speed of model 208 using the sparse convolutions is much faster than that using the traditional convolutions in terms of orders of magnitude.

FIG. 6 is a graphical representation illustrating another exemplary application of sparse-convolution-based model 208 for a branch label prediction, according to certain embodiments of the disclosure. Similar to FIG. 5, a centerline of a coronary artery in a cardiac image can be extracted and discretized to generate centerline points. Sparse data can be generated to include centerline points of the coronary artery. Sparse-convolution-based model 208 can be applied to the cardiac image and the sparse data to generate a label prediction result.

For example, image processing device 103 may generate an input sparse tensor including the sparse data (e.g., a set of elements corresponding to the centerline points of the coronary artery) and a set of input features corresponding to the centerline points of the coronary artery. The input features may be received via a user interface or automatically generated by feature extraction network 212 from the cardiac image. Sparse-convolution-based model 208 (e.g., prediction network 214 of model 208) may be applied to the cardiac image and the input sparse tensor to generate the label prediction result.

Consistent with certain embodiments of the present disclosure, sparse-convolution-based model 208 can be applied in the FFR prediction and the branch label prediction as shown in FIGS. 5-6. It is understood that sparse-convolution-based model 208 is also applicable to any other biomedical predictions such as disease progression prediction, disease severity prediction, follow-up condition prediction, etc.

FIG. 7 is a flowchart of an exemplary method 700 for training a sparse-convolution-based model, according to certain embodiments of the disclosure. As shown in FIG. 7, the method may begin, at step S702, with establishing a training database. For example, model training device 102 may establish training database 101 to include different sets of training data. The different sets of training data may be annotated manually or automatically, and ground truth values (or ground truth annotations) can be provided for the different sets of training data manually or automatically.

The method may also include, at step S704, retrieving one or more sets of training data from the training database. For example, model training device 102 may retrieve one or more sets of training data from training database 101.

The method may further include, at step S706, training the sparse-convolution-based model using the one or more sets of training data. For example, model training device 102 may train sparse-convolution-based model 208 based on the one or more sets of training data retrieved from training database 101.

The method may further include, at step S708, determining whether an objective training function converges. For example, model training device 102 may determine whether an objective training function (e.g., a loss function) of sparse-convolution-based model 208 converges based on an optimization method such as SGD, RMSProp, Adam, or the like. If the objective training function converges, the method may proceed to step S710. Otherwise, the method may return to step S704 to continue training the sparse-convolution-based model. In some embodiments, the method may continue training the sparse-convolution-based model until the objective training function converges.

The method may additionally include, at step S710, storing the trained sparse-convolution-based model. For example, model training device 102 may store a structure and parameters of the trained sparse-convolution-based model in a storage associated with device 102 or a storage in the cloud. Model training device 102 may also provide the trained sparse-convolution-based model to image processing device 103 for later use.

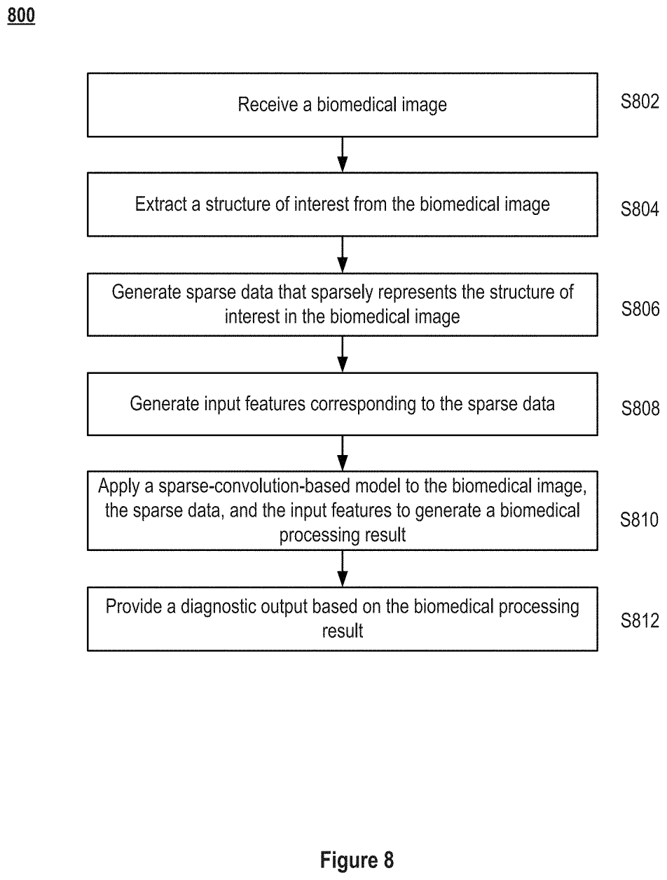

FIG. 8 is a flowchart of an exemplary method 800 for representation learning with at least a sparse convolution, according to certain embodiments of the disclosure. As shown in FIG. 8, the method may begin, at step S802, with receiving a biomedical image. For example, communication interface 302 of image processing device 103 may receive a biomedical image acquired by image acquisition device 105. The biomedical image can be a new biomedical image to be processed.

The method may also include, at step S804, extracting a structure of interest from the biomedical image. For example, processor 308 of image processing device 103 may extract a structure of interest from the biomedical image.

The method may also include, at step S806, generating sparse data that sparsely represents the structure of interest in the biomedical image. For example, processor 308 may generate sparse data that includes a set of coordinates for a set of elements sparsely representing the structure of interest. In some embodiments, the structure of interest can be a centerline of a blood vessel, and the sparse data may include centerline points of the blood vessel.

The method may also include, at step S808, generating input features corresponding to the sparse data. For example, processor 308 may generate the input features for the set of elements included in the sparse data. In some embodiments, processor 308 may apply a feature extraction network based on the set of coordinates of the sparse data to extract the input features from the biomedical image.

The method may further include, at step S810, applying a sparse-convolution-based model to the biomedical image, the sparse data, and the input features to generate a biomedical processing result. For example, processor 308 may apply a sparse-convolution-based model to the biomedical image and an input sparse tensor (including the sparse data and the input features) to generate a biomedical processing result for the biomedical image. The sparse-convolution-based model may perform one or more neural networks including the sparse convolution on the input sparse tensor.

In some embodiments, the biomedical processing result may include processing features for the biomedical image, and the sparse-convolution-based model (or another suitable model such as a RNN model) can be used to generate a biomedical prediction result from the biomedical image based on the processing features and the sparse data.

In some embodiments, the biomedical processing result may include a biomedical prediction result for the biomedical image. For example, the sparse-convolution-based model may include a prediction network. Processor 308 may apply the prediction network to generate a biomedical prediction result based on the biomedical image and the input sparse tensor.

The method may additionally include, at step S812, providing a diagnostic output based on the biomedical processing result. For example, processor 308 may generate and output a diagnostic result based on the biomedical processing result. The diagnostic output may include any suitable diagnostic analysis associated with a patient, such as a disease type, severity of the disease, a predicted progression of the disease, potential treatments for the disease, a suggested follow-up examination, or the like, associated with the patient.

For example, the biomedical processing result may include an FFR prediction result for a coronary artery of the patient. Based on the FFR prediction result, processor 308 may generate a diagnostic output including a diagnosis of a coronary heart disease, diagnostic suggestions on assessing whether or not to perform angioplasty or stenting on blockages of the coronary artery, or the like.

According to certain embodiments, a non-transitory computer-readable medium may have a computer program stored thereon. The computer program, when executed by at least one processor, may perform a method for biomedical image analysis. For example, any of the above-described methods may be performed in this way.

While the disclosure uses biomedical images as examples that the disclosed systems and methods are applied to analyze, it is contemplated that the disclosed representation learning with a sparse convolution can be applied to other types of images beyond biomedical images. The images can capture any object, scene, or structures, and an ordinary skill in the art will appreciate that the disclosed systems and methods can be readily adapted to analyze these other images.

In some embodiments, the computer-readable medium may include volatile or non-volatile, magnetic, semiconductor, tape, optical, removable, non-removable, or other types of computer-readable medium or computer-readable storage devices. For example, the computer-readable medium may be the storage device or the memory module having the computer instructions stored thereon, as disclosed. In some embodiments, the computer-readable medium may be a disc or a flash drive having the computer instructions stored thereon.

It will be apparent to those skilled in the art that various modifications and variations can be made to the disclosed system and related methods. Other embodiments will be apparent to those skilled in the art from consideration of the specification and practice of the disclosed system and related methods.

It is intended that the specification and examples be considered as exemplary only, with a true scope being indicated by the following claims and their equivalents.

What is claimed is:

1. A system for representation learning from a biomedical image with a sparse convolution, comprising:
a communication interface configured to receive the biomedical image acquired by an image acquisition device; and
at least one processor, configured to:
extract a structure of interest from the biomedical image;
generate sparse data representing the structure of interest and input features corresponding to the sparse data; and
apply a sparse-convolution-based model to the biomedical image, the sparse data, and the input features to generate a biomedical processing result for the biomedical image, wherein the sparse-convolution-based model performs one or more neural network operations comprising the sparse convolution on the sparse data and the input features.

2. The system of claim 1, wherein the at least one processor is further configured to:
provide a diagnostic output based on the biomedical processing result.

3. The system of claim 1, wherein the sparse data comprises a set of coordinates for a set of elements sparsely representing the structure of interest, and wherein each input feature corresponds to an element in the set of elements.

4. The system of claim 3, wherein the at least one processor is further configured to:
generate an input sparse tensor including the sparse data representing the structure of interest and the input features corresponding to the sparse data,
wherein the sparse-convolution-based model is applied to the biomedical image and the input sparse tensor to generate the biomedical processing result.

5. The system of claim 3, wherein the at least one processor is further configured to:
receive or automatically generate the input features corresponding to the set of elements.

6. The system of claim 5, wherein the sparse-convolution-based model comprises an input feature extraction network, and to automatically generate the input features, the at least one processor is further configured to:
apply the input feature extraction network based on the set of coordinates to extract the input features from the biomedical image.

7. The system of claim 1, wherein the biomedical processing result comprises processing features corresponding to the sparse data, and wherein the sparse-convolution-based model is applied to extract the processing features from the biomedical image based on the sparse data and the input features.

8. The system of claim 7, wherein the sparse-convolution-based model is further applied to generate a biomedical prediction result from the biomedical image based on the sparse data and the processing features.

9. The system of claim 7, wherein the at least one processor is further configured to:
apply a prediction model to generate a biomedical prediction result from the biomedical image based on at least one of the sparse data or the processing features.

10. The system of claim 1, wherein:
the biomedical processing result comprises a biomedical prediction result; and
the sparse-convolution-based model is applied to generate the biomedical prediction result from the biomedical image based on the sparse data and the input features.

11. The system of claim 1, wherein the structure of interest is a centerline of a blood vessel, and wherein the sparse data comprises centerline points of the blood vessel.

12. The system of claim 11, wherein the biomedical processing result comprises at least one of a fractional flow reserve (FFR) prediction result or an instantaneous wave-free ratio (iFR) prediction result for the blood vessel.

13. The system of claim 12, wherein the FFR prediction result comprises an FFR value at a centerline point or a relative FFR change at the centerline point compared to a neighboring point.

14. The system of claim 12, wherein the iFR prediction result comprises an iFR value at a centerline point or a relative iFR change at the centerline point compared to a neighboring point.

15. The system of claim 11, wherein the biomedical processing result comprises a label prediction result for the blood vessel.

16. The system of claim 15, wherein the label prediction result comprises at least one of a label of a main branch or a label of a side-branch in the blood vessel.

17. The system of claim 11, wherein the biomedical image is a cardiac image, wherein the blood vessel is a coronary artery, and wherein the centerline points form a coronary artery tree.

18. The system of claim 1, wherein the sparse-convolution-based model includes at least one of a Convolutional Neural Network (CNN), a Multilayer Perceptron (MLP), a Fully Convolutional Network (FCN), a tree structured recurrent neural network (RNN), or a graph network.

19. A computer-implemented method for representation learning from a biomedical image with a sparse convolution, comprising:
receiving, at a communication interface, the biomedical image acquired by an image acquisition device;
extracting, by at least one processor, a structure of interest from the biomedical image;
generating, by the at least one processor, sparse data representing the structure of interest and input features corresponding to the sparse data; and
applying, by the at least one processor, a sparse-convolution-based model to the biomedical image, the sparse data, and the input features to generate a biomedical processing result for the biomedical image, wherein the sparse-convolution-based model performs one or more neural network operations comprising the sparse convolution on the sparse data and the input features.

20. A non-transitory computer-readable medium having a computer program stored thereon, wherein the computer program, when executed by at least one processor, performs a method for representation learning from a biomedical image with a sparse convolution, the method comprising:
receiving the biomedical image acquired by an image acquisition device;
extracting a structure of interest from the biomedical image;
generating sparse data representing the structure of interest and input features corresponding to the sparse data; and
applying a sparse-convolution-based model to the biomedical image, the sparse data, and the input features to generate a biomedical processing result for the biomedical image, wherein the sparse-convolution-based model performs one or more neural network operations comprising the sparse convolution on the sparse data and the input features.
